(19) United States
(12) Patent Application Publication
(10) Pub. No.: US 2026/0319042 A1
(43) Pub. Date: Oct. 15, 2026

(54) CARDIAC IMAGING

(71) Applicants: Siemens Healthineers AG, Forchheim (DE); KING'S COLLEGE LONDON, London (GB)

(72) Inventors: Radhouene NEJI, London (GB); Oscar GOLDMAN, London (GB); Karl-Philipp KUNZE, London (GB); John WHITAKER, Hither Green (GB); Amedeo CHIRIBIRI, London (GB)

(73) Assignees: Siemens Healthineers AG, Forchheim (DE); KING'S COLLEGE LONDON, London (GB)

(21) Appl. No.: 19/644,445

(22) Filed: Apr. 10, 2026

(30) Foreign Application Priority Data
Apr. 11, 2025 (EP) 25169940.1

(51) Int. Cl. G01R 33/48; A61B 5/00; A61B 5/055; G01R 33/56; G01R 33/561; G01R 33/567

(52) U.S. Cl. CPC G01R 33/4818; A61B 5/0044; A61B 5/055; G01R 33/5602; G01R 33/5612; G01R 33/5616; G01R 33/567

(57) ABSTRACT

A method for generating MR images of a patient at an MR imaging system comprises sampling a k-space with a 3-dimensional acquisition schema of k-space with two phase encoding directions and one read out direction. The sampling schema includes applying a sequence of inversion pulses to the patient to invert a magnetization of the patient, wherein sampling of the k-space is performed with a uniform undersampling of the k-space and n different EPI readout shots in the read-out direction after each inversion pulse, with n being ≥5. The MR images are reconstructed based on the sampled k-space.

### CROSS-REFERENCE TO RELATED APPLICATION(S)

The present application claims priority under 35 U.S.C. § 119 to European Patent Application No. 25169940.1, filed Apr. 11, 2025, the entire contents of which is incorporated herein by reference.

### FIELD

One or more example embodiments of the present invention relate to a method for generating MR images of a patient using a 3 dimensional acquisition schema of k-space, to a Magnetic Resonance imaging (MRI) system, a computer program comprising program code, and a carrier comprising the computer program. One or more example embodiments of the present invention especially relate to cardiac imaging.

### BACKGROUND

Cardiac imaging with MRI has emerged as a valuable diagnostic tool in the assessment of cardiovascular disease. MRI offers high-resolution images of the heart and its surrounding structures, allowing for detailed evaluation of cardiac anatomy, function, and pathology. MRI imaging of the heart provides high spatial resolution together with excellent soft tissue contrast and functional assessment. Late Gadolinium Enhanced (LGE) MRI is a key component of cardiac MRI exams. It is used to detect and classify myocardial infarction. 3D LGE acquisitions can suffer however from long scan times, which may be difficult for some patients to tolerate. A recent proposal to address this was to produce 3D LGE images in a single breath-hold using Compressed-Sensing (CS) as discussed in Roman Johannes Gertz et al. “Compressed SENSE accelerated 3D single-breathhold late gadolinium enhancement cardiovascular magnetic resonance with isotropic resolution: clinical evaluation”. In: Frontiers in Cardiovascular Medicine 10 (November 2023). This was achieved by acquiring highly undersampled images; reducing scan time. However, CS reconstructions can suffer from residual blurring artifacts, particularly at higher undersampling factors.

Accordingly a need exists to avoid the drawbacks mentioned above and to provide an imaging method with reduced artifacts and acceptable image acquisition time.

### SUMMARY

This need is met by the features of the independent claims. Further aspects are described in the dependent claims.

According to a first aspect a method for generating MR images of a patient at an MR imaging system is provided the method comprising the step of sampling a k-space with a three-dimensional acquisition scheme of k-space with two phase encoding directions and one readout direction wherein the sampling includes an application of a sequence of inversion pulses to the patient to invert a magnetization of the patient and the sampling of the k-space is performed with a uniform undersampling of the k-space and n different EPI readout shots in the readout direction after each inversion pulse with n>5. The MR images are then reconstructed based on the sampled k-space.

The combination of the uniform undersampling, the inversion pulses and the EPI readout after each inversion pulse, provides a very effective sampling with reduced acquisition time and acceptable image quality.

Preferably, the MR images are MR images of the heart and the three-dimensional acquisition scheme is used with cardiac gating wherein the n readout shots are acquired within one heartbeat. The imaging method provides especially good results in cardiac imaging and the acquisition of the MR images is possible within a single breath-hold.

Preferably the sampling of k-space is carried out after a contrast agent has been applied to the patient and a concentration of the contrast agent has reached an equilibrium in the patient. This is also known as late contrast agent enhanced imaging, also called Late Gadolinium Enhanced, LGE, imaging which is especially useful for certain findings in the heart such as the detection and classification of myocardial infarction. The proposed image acquisition scheme is especially useful in this field of application.

Preferably the k-space is divided in one of the phase encoding directions into n regions and one EPI readout shot from the n shots is acquired from each region within one heartbeat. As a consequence, a uniform undersampling is obtained which is helpful for the image reconstruction.

Additionally, it is possible that in each of the n regions of the k-space S shots are acquired with S>=3 and the k-space trajectories of shots within one region are shifted relative to one another in at least one of the phase encoding directions using a CAIPIRINHA imaging technique. Here S is preferably in the range between 6 and 10. With this image acquisition and reconstruction technology, it is possible to acquire a 3D whole heart volume during a single breath-hold with satisfying image quality and reduced artifacts. Preferably reconstructing the MR images is performed using a k-space interpolation based technique including GRAPPA (GeneRalized Autocalibrating Partial Parallel Acquisition) using 3D calibration kernels.

Furthermore, the n EPI readout shots can be distributed in k-space such that a center of the k-space is sampled with earlier shots from the n shots and areas outside the center are sampled with later shots from the n shots. The center of k-space which is responsible for the contrast in the image is acquired earlier at a larger magnetization and thus MR signal. As a consequence, the image contrast is improved when the center of k-space is sampled earlier compared to the outer parts of k-space.

Preferably one shot from the n shots is acquired in each of the n regions after each inversion pulse and n is in the range between 5 and 10, more preferably in the range between 6 and 8.

Preferably the inversion time is selected in dependence on an MR signal of the myocardium and more preferably it is selected such that the MR signal of the myocardium is close to zero at image acquisition so that a good contrast is obtained in the images in the area around the myocardium.

Furthermore, an MR imaging system is provided comprising a processing unit and a memory configured to store program code which when carried out by the processing unit causes the processing unit to carry out the above identified steps of acquiring an image.

Furthermore, a computer program comprising program code is provided to be executed by a processing unit of an MR imaging system wherein execution of the program code causes the processing unit to carry out a method as discussed above or as discussed in further detail below.

Finally, a carrier comprising the computer program is provided wherein the carrier is one of an electronic signal, optical signal, radio signal, and computer-readable storage medium.

It is to be understood that the features mentioned above and features yet to be explained below can be used not only in the respective combinations indicated, but also in other combinations or in isolation without departing from the scope of the present invention. Features of the above-mentioned aspects and embodiments described below may be combined with each other in other embodiments unless explicitly mentioned otherwise.

### BRIEF DESCRIPTION OF THE DRAWINGS

FIG. 1 is a schematic view of an MRI imaging system with which a 3 D whole heart volume can be acquired within a single breath-hold.

FIG. 2 shows a schematic view of part of a sampling schema used for acquiring a 3D volume of the heart

FIG. 3 shows a schematic view of a sampling pattern used to sample k-space during the sampling schema of FIG. 2.

FIG. 4 shows an example schematic view of a flowchart comprising steps carried out during the image acquisition of the k-space sampling.

### DETAILED DESCRIPTION

In the following, embodiments of the present invention will be described in detail with reference to the accompanying drawings. It is to be understood that the following description of embodiments is not to be taken in a limiting sense. The scope of the present invention is not intended to be limited by the embodiments described hereinafter or by the drawings, which are to be illustrative only.

The drawings are to be regarded as being schematic representations, and elements illustrated in the drawings are not necessarily shown to scale. Rather, the various elements are represented such that their function and general purpose become apparent to a person skilled in the art. Any connection or coupling between functional blocks, devices, components of physical or functional units shown in the drawings and described hereinafter may be implemented by an indirect connection or coupling. A coupling between components may be established over a wired or wireless connection. Functional blocks may be implemented in hardware, software, firmware, or a combination thereof.

Independent of the grammatical term usage, individuals with male, female or other gender identities are included within the term.

In the following, an MR imaging method will be discussed which provides a 3D image data set of the heart which is short enough that it can be acquired in a single breath-hold which does not suffer from artificial image appearance and residual blurring.

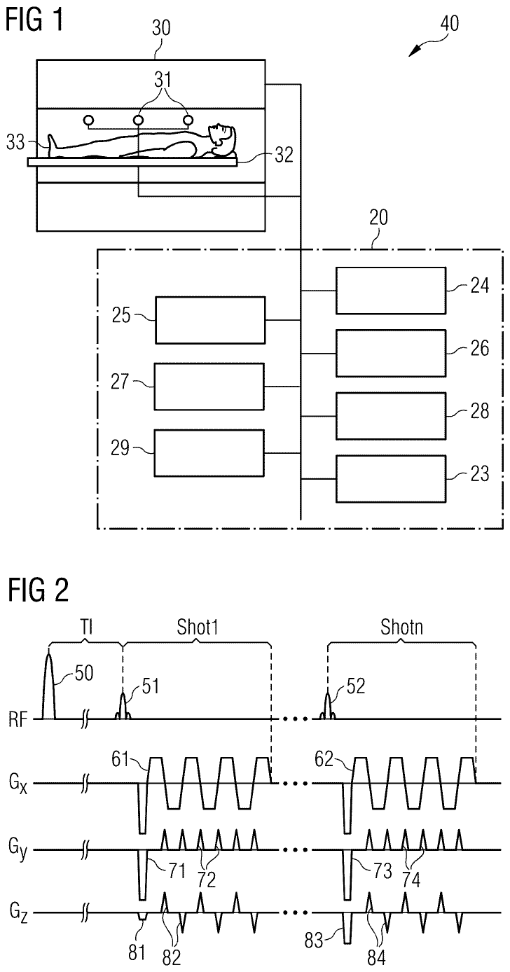

FIG. 1 shows a schematic view of an MRI (Magnetic Resonance Imaging) system 40, which comprises a magnet 30 generating a polarization field B0. An object 33 under examination lying on a table 32 is moved into the center of the MRI system 40 where MR signals after RF excitation can be detected by a receiving coil 31, which can comprise different coil sections wherein each coil section is associated with a corresponding detection channel. By applying RF pulses and magnetic field gradients, the nuclear spins in the object 33, especially in the part located in the receiving coil 31 are excited and the currents induced by the relaxation are detected. The way how MR images are generated and how the MR signals are detected using a sequence of RF pulses and the sequence of magnetic field gradients are known in the art so that a detailed explanation thereof is omitted.

The MRI system comprises a control unit 20 which is used for controlling the MRI system. The control unit 20 comprises a gradient control unit 24 for controlling and switching the magnetic field gradients, an RF control unit 25 for controlling and generating the RF pulses for the imaging sequences. An image sequence control unit 26 is provided which controls the sequence of the applied RF pulses and magnetic field gradients and thus controls the gradient control unit 24 and the RF control unit 25. In a memory 27, computer programs needed for operating the MRI system and the imaging sequences necessary for generating the MR images can be stored together with the generated MR images. The generated MR images can be displayed on a display 28 wherein input unit 29 is provided used by a user of the MR system to control the functioning of the MRI system. A processing unit 23 can coordinate the operation of the different functional units shown in FIG. 1 and can comprise one or more processors which can carry out instructions stored on the memory 27. The memory includes the program code to be executed by the processing unit 23.

FIG. 2 shows a structure of a sequence diagram which can be used to generate a 3D data set during a single breath-hold. The imaging sequence uses an inversion recovery-prepared segmented 3D EPI sequence with a skipped-CAIPIRINHA undersampling pattern. An inversion pulse 50 is applied and after the inversion pulse n shots with shorter excitation pulses 51 and 52 are applied to excite the magnetization and after each shot an echo planar imaging, EPI like data acquisition and gradient switching in the readout direction is applied as shown by the gradient switching 61 and 62. The gradient switching in the readout direction (here Gx) generates gradient echos acquired during the switching of Gx. In the phase-encoding directions Gy and Gz a CAIPIRINHA undersampling pattern is used including the gradient switchings 71, 72, 73 and 74 in one of the phase encoding directions together with the gradient switchings 81-84 in the other phase-encoding direction.

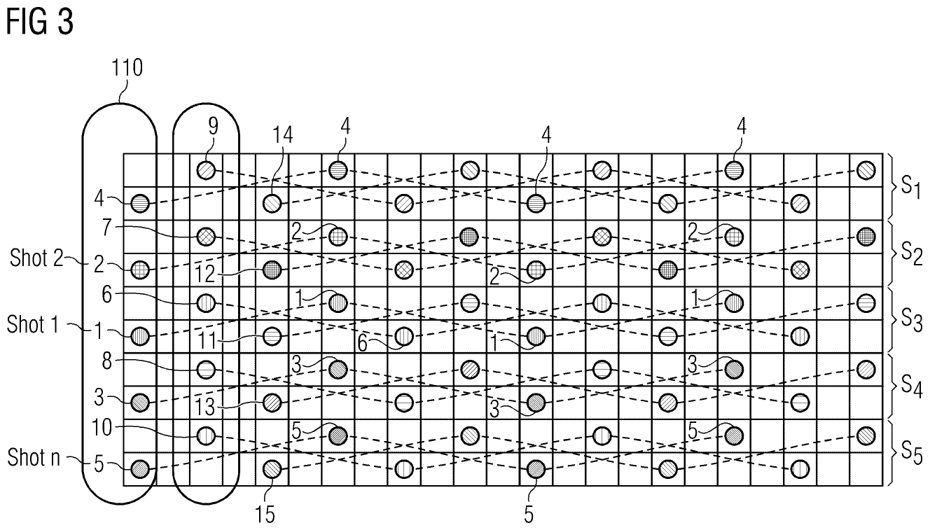

Referring also to FIG. 3, the sampling k-space pattern is shown in more detail. A CAIPIRINHA on the sampling pattern was used and in this scheme a set of several parameters parametrize the sampling mask. The parameter Ry describes the undersampling factor in the phase encoding direction, the parameter Rz describes the undersampling factor in the partition encoding direction and Δ describes the partition offset between adjacent samples respectively.

In FIG. 3 the numbers shown indicate an order of k-space sampling points wherein all points with number “1” belong to the sampling after the first of the n inversion pulses and all numbers “2” belong to the second data acquisition after the second inversion pulse 52 of FIG. 2. The fields containing a number include a sample wherein the other empty fields were not sampled and the readout direction is running perpendicular to the image plane. Furthermore, the k-space is segmented into several n segments (here S1 to S5) and describes the segmentation factor which determines how the sampling mask is segmented into EPI shots. In the example shown each of the segments S1 to S5 contain three different data acquisitions, however it should be understood that this is only for illustration purposes. Accordingly, in the example indicated in FIG. 3 S is 3 and in each of the segments 3 data acquisition trains are acquired. The image acquisition is then further segmented between different cardiac cycles, here the R-R interval introducing the other parameter n, the number of shots played out after each inversion pulse. This number n is 5 in the example shown in FIG. 3 and the segmentation divides the k-space into n regions, here the five regions and one shot from each region is acquired after each inversion pulse starting from the most central region and moving outward in a centric pattern as shown by the numbers 2, 3, 4 and 5. Accordingly, within each inversion pulse the shots indicated by region 110 are acquired so region 110 includes the number n of shots acquired within one heartbeat. Accordingly, the image acquisition is described by five parameters, Ry, Rz, Δ, S and n which determine the order in which the k-space points are acquired. The image acquisition was tested in patients using a 16 channel coil and a 32 channel spine coil. The imaging sequence was acquired with the following parameters:
a transverse orientation, 2.5 mm isotropic resolution providing a matrix of 128 128 56 and a field of view of 320 320 140 mm³. Echo spacing was 0.68 ms, the receiver bandwidth was 1964 Hz/px, a 20° flip angle was used and 1-2-1 binomial water excitation pulse was used for fat suppression. The parameters for (Ry, Rz, Δ, S, n) were (2, 2, 1, 8, 7) providing an echo train length of 16, an acquisition window of 119 ms and a total undersampling factor of 4. A full 3D heart image could be acquired over 19 heartbeats, such as one R-R interval.

For the LGE acquisitions shown in FIGS. 2 and 3, the inversion time was chosen to null viable myocardium. Furthermore, it is possible to choose a trigger delay such that the acquisition window for an acquisition of the MR signal after the inversion pulse is placed in the quiescent period of the heart, such as the diastole. The image reconstruction could be carried out using an iterative SENSE reconstruction with a stopping criterion being a residual below a predefined threshold fixed to a certain threshold value e.g. 10⁻⁶, or any other value between 10⁻⁸ and 10⁻⁴ The coil sensitivity maps used for the reconstruction can be generated from an external low resolution free breathing gradient echo reference scan, which could preferably be normalized with a body coil.

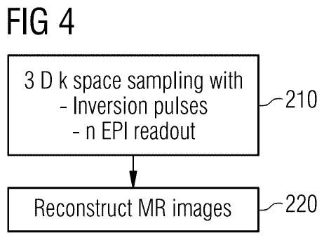

FIG. 4 summarizes some of the steps used for the image acquisition discussed above in connection with FIGS. 2 and 3. In step 210 a 3D k-space sampling is carried out using the 2 phase-encoding directions and one readout direction in which a sequence of inversion pulses is applied to invert a magnetization and n different EPI readout shots are used in the readout direction after each inversion pulse. In step 220 the MR images are then reconstructed based on the sampled k-space.

Summarizing the above discussed method enables a fast acquisition of images which allows a 3D single breath-hold image acquisition without the need for very high undersampling factors.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternatively, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” or “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different from that of the methods described, and/or components such as the described system, architecture, devices, circuit, and the like, may be connected or combined to be different from the above-described methods, or results may be appropriately achieved by other components or equivalents.

What is claimed is:

1. A method for generating MR images of a patient at an MR imaging system, the method comprising:
sampling a k-space with a 3-dimensional acquisition schema of k-space with two phase encoding directions and a read out direction, wherein sampling of the k-space includes applying a sequence of inversion pulses to the patient to invert a magnetization of the patient, wherein sampling of the k-space is performed with a uniform undersampling of the k-space and n different EPI readout shots in the read out direction after each inversion pulse, and wherein n is ≥5; and
reconstructing the MR images based on the sampled k-space.

2. The method of claim 1, wherein
the MR images are MR images of a heart,
the 3-dimensional acquisition schema is used with cardiac gating, and
the n different EPI readout shots are acquired within one heartbeat.

3. The method of claim 2, wherein
the k-space is divided, in one of the two phase encoding directions, into n regions, and
one EPI readout shot, from the n different EPI readout shots, is acquired from each region within one heartbeat.

4. The method of claim 3, wherein
in each of the n regions of the k-space, S shots are acquired, with S being ≥3, and
k-space trajectories of shots within one region are shifted relative to one another in one of the two phase encoding directions using a CAIPIRINHA imaging technique.

5. The method of claim 4, wherein S is in a range between 6 and 10.

6. The method of claim 4, wherein reconstructing of the MR images is performed using a k-space interpolation based technique including GRAPPA using 3D calibration kernels.

7. The method of claim 1, wherein sampling of the k-space is carried out after a contrast agent has been applied to the patient and a concentration of the contrast agent has reached an equilibrium in the patient.

8. The method of claim 1, wherein the n different EPI readout shots are distributed in k-space such that a center of the k-space is sampled with earlier shots from the n different EPI readout shots and areas outside the center are sampled with later shots from the n different EPI readout shots.

9. The method of claim 3, wherein one shot from the n different EPI readout shots is acquired in each of the n regions after each inversion pulse.

10. The method of claim 1, wherein n is between 5 and 10.

11. The method of claim 2, wherein an inversion time is selected based on an MR signal of a myocardium.

12. An MR imaging system comprising:
at least one processor; and
a memory configured to store program code, which when executed by the at least one processor, causes the MR imaging system to
sample a k-space with a 3-dimensional acquisition schema of k-space with two phase encoding directions and a read out direction, wherein sampling of the k-space includes applying a sequence of inversion pulses to a patient to invert a magnetisation of the patient, wherein sampling of the k-space is performed with a uniform undersampling of the k-space and n different EPI readout shots in the read out direction after each inversion pulse, and wherein n is ≥5, and
reconstruct MR images based on the sampled k-space.

13. An MR imaging system comprising:
at least one processor; and
a memory configured to store program code, which when executed by the at least one processor, causes the MR imaging system to perform the method of claim 2.

14. A non-transitory computer-readable medium storing computer-executable instructions that, when executed by at least one processor at an MR imaging system, cause the MR imaging system to perform the method of claim 1.

15. The method of claim 8, wherein
the k-space is divided, in one of the two phase encoding directions, into n regions,
one EPI readout shot, from the n different EPI readout shots, is acquired from each region within one heartbeat, and
one shot from the n different EPI readout shots is acquired in each of the n regions after each inversion pulse.

16. The method of claim 2, wherein an inversion time for the sequence of inversion pulses is selected such that an MR signal of myocardium is substantially zero during MR signal acquisition.

17. The method of claim 5, wherein reconstructing of the MR images is performed using a k-space interpolation based technique including GRAPPA using 3D calibration kernels.

18. The method of claim 2, wherein sampling of the k-space is carried out after a contrast agent has been applied to the patient and a concentration of the contrast agent has reached an equilibrium in the patient.

19. The method of claim 2, wherein the n different EPI readout shots are distributed in k-space such that a center of the k-space is sampled with earlier shots from the n different EPI readout shots and areas outside the center are sampled with later shots from the n different EPI readout shots.

20. The method of claim 5, wherein the n different EPI readout shots are distributed in k-space such that a center of the k-space is sampled with earlier shots from the n different EPI readout shots and areas outside the center are sampled with later shots from the n different EPI readout shots.
